(19) United States
(12) Patent Application Publication
(10) Pub. No.: US 2026/0240834 A1
(43) Pub. Date: Aug. 20, 2026

(54) METHODS OF TREATING CANCER

(71) Applicant: XRad Holding Cayman Limited, Grand Cayman (KY)

(72) Inventors: Michael KASTAN, Chapel Hill, NC (US); David KIRSCH, Durham, NC (US); Tona GILMER, Rougemont, NC (US)

(21) Appl. No.: 19/162,482

(22) PCT Filed: Mar. 5, 2024
(86) PCT No.: PCT/US2024/018499

§ 371 Date: Sep. 5, 2025

Related U.S. Application Data
(60) Provisional application No. 63/488,647, filed on Mar. 6, 2023.

(51) Int. Cl. A61K 31/4745; A61K 31/454; A61K 31/502; A61K 31/5025; A61K 31/55; A61P 35/00

(52) U.S. Cl. CPC A61K 31/4745; A61K 31/454; A61K 31/502; A61K 31/5025; A61K 31/55; A61P 35/00

(57) ABSTRACT

Disclosed are methods of treating a cancer in a subject in need thereof using a dual ATM and DNA-PK inhibitor optionally in combination with a PARP inhibitor.

### FIELD OF THE INVENTION

The invention relates to the use of dual ATM and DNA-PK inhibitors, pharmaceutically acceptable salts thereof, or pharmaceutical compositions containing the same, in treating homologous recombination-deficient cancer. The dual ATM and DNA-PK inhibitors may be used alone or in combination with PARP inhibitors, pharmaceutically acceptable salts thereof, or pharmaceutical compositions containing the same.

### BACKGROUND OF THE INVENTION

Several members of the PIKK (PI-3K-like Kinase) family of serine-threonine kinases are known mediators of DNA damage signaling.

Radiation therapy (RT) is used to treat >50% of all cancer patients at some point during their illness. Despite significant effort, previous approaches to develop clinical radiosensitizers have not been highly effective, primarily as a result of targeting non-specific pathways which are not direct regulators of the cellular response to radiation.

Inhibitors of poly (ADP-ribose) polymerases (PARP inhibitors) target the DNA repair enzyme poly (ADP-ribose) polymerase 1 (PARP1) and closely related paralogs. Several PARP inhibitors (olaparib, niraparib, rucaparib, talazoparib) have been approved for treatment of various cancers (e.g., ovarian cancer, breast cancer, fallopian tube cancer, and primary peritoneal cancer).

There is a need for new therapies for oncological diseases, particularly, effective treatments for homologous recombination-deficient cancers.

### SUMMARY OF THE INVENTION

In general, the invention provides methods of treating cancer using a dual ATM and DNA-PK inhibitor, or a pharmaceutically acceptable salt thereof. The methods may involve treating a homologous recombination-deficient (HR-deficient) cancer. Examples of HR-deficient cancers include those having a loss of function BRCA (e.g., BRCA1 or BRCA2) mutation. HR-deficient cancers, such as those with a loss of function BRCA mutation, may be sensitized to treatment with dual ATM and DNA-PK inhibitors as a single agent. In some embodiments, further benefit may be achieved when the dual ATM and DNA-PK inhibitors are used as part of a combination therapy with a PARP inhibitor, or a pharmaceutically acceptable salt thereof.

In one aspect, the invention provides a method of treating a homologous recombination (HR)-deficient cancer in a subject. The method includes administering to the subject in need thereof a therapeutically effective amount of a dual ATM and DNA-PK inhibitor.

In some embodiments, the HR-deficient cancer is a BRCA-mutant cancer. In some embodiments, the cancer has a loss of function BRCA mutation. In some embodiments, the cancer has been previously identified as a cancer having a loss of function BRCA mutation.

In a further aspect, the invention provides a method of treating cancer in a subject, the method including:
(i) identifying the cancer as being an HR-deficient cancer; and
(ii) administering to the subject in need thereof a therapeutically effective amount of a dual ATM and DNA-PK inhibitor.

In yet another aspect, the invention provides a method of inducing cell death in an HR-deficient cancer cell, the method comprising contacting the cell with an effective amount of a dual ATM and DNA-PK inhibitor.

In some embodiments of any of the methods described herein, the HR-deficient cancer has a loss of function of BRCA1 or BRCA2, or a combination thereof.

In a further aspect, the invention provides a method of treating a homologous recombination (HR)-deficient cancer in a subject, the method including administering to the subject in need thereof a therapeutically effective amount of a dual ATM and DNA-PK inhibitor and a therapeutically effective amount of a PARP inhibitor.

In some embodiments, the HR-deficient cancer is a BRCA-mutant cancer. In some embodiments, the cancer has a loss of function BRCA mutation. In some embodiments, the cancer has been previously identified as a cancer having a loss of function BRCA mutation.

In still another aspect, the invention provides a method of treating cancer in a subject, the method including:
(i) identifying the cancer as being an HR-deficient cancer; and
(ii) administering to the subject in need thereof a therapeutically effective amount of a dual ATM and DNA-PK inhibitor and a therapeutically effective amount of a PARP inhibitor.

In another aspect, the invention provides method of inducing cell death in an HR-deficient cancer cell. The method includes contacting the cell with an effective amount of a dual ATM and DNA-PK inhibitor and a therapeutically effective amount of a PARP inhibitor.

In some embodiments of the methods described herein, the HR-deficient cancer has a loss of function of BRCA1 or BRCA2, or a combination thereof.

In some embodiments of any of the methods described herein, the cancer has a BRCA1 mutation. In some embodiments of any of the methods described herein, the cancer has a BRCA2 mutation.

In some embodiments of the combination therapies described herein, the dual ATM and DNA-PK inhibitor is administered before the PARP inhibitor. In some embodiments, the dual ATM and DNA-PK inhibitor is administered after the PARP inhibitor. In some embodiments, the dual ATM and DNA-PK inhibitor is co-administered with the PARP inhibitor.

In some embodiments, the PARP inhibitor is olaparib or a pharmaceutically acceptable salt thereof, niraparib or a pharmaceutically acceptable salt thereof, rucaparib or a pharmaceutically acceptable salt thereof, or talazoparib or a pharmaceutically acceptable salt thereof. In some embodiments, the PARP inhibitor is niraparib or a pharmaceutically acceptable salt thereof. In some embodiments, the PARP inhibitor is olaparib or a pharmaceutically acceptable salt thereof. In some embodiments, the PARP inhibitor is rucaparib or a pharmaceutically acceptable salt thereof. In some embodiments, the PARP inhibitor is talazoparib or a pharmaceutically acceptable salt thereof.

In some embodiments of any of the foregoing aspects, the dual ATM and DNA-PK inhibitor is a compound of formula (I):

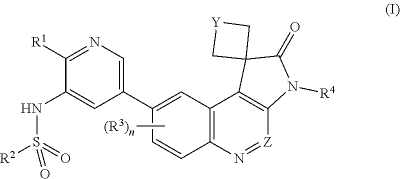

or a pharmaceutically acceptable salt thereof,
wherein
Y is CHR⁵ or NR⁶;
Z is CH, CR³, or N;
n is 0, 1, 2, or 3;
R¹ is-O-L-N(R⁷)₂ or optionally substituted, four-memberred, saturated N-heterocyclyl;
R² is C₁₋₃ alkyl;
each R³ is independently halogen or optionally substituted C₁₋₃ alkyl;
R⁴ is optionally substituted alkyl;
R⁵ is hydrogen, optionally substituted C₁₋₃ alkyl, or benzyloxy;
R⁶ is optionally substituted C₁₋₃ alkyl;
each R⁷ is independently H or optionally substituted C₁₋₃ alkyl; and
L is optionally substituted ethylene.

In some embodiments, the dual ATM and DNA-PK inhibitor of Formula II is a compound of formula (IA):

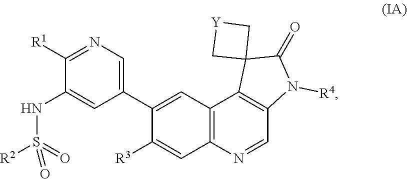

or a pharmaceutically acceptable salt thereof.

In some embodiments, the dual ATM and DNA-PK inhibitor of Formula II is a compound of formula (IB):

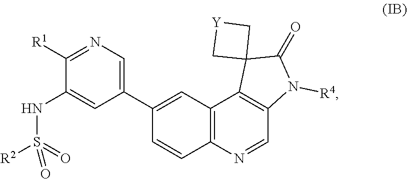

or a pharmaceutically acceptable salt thereof.

In some embodiments, the dual ATM and DNA-PK inhibitor is a compound selected from:

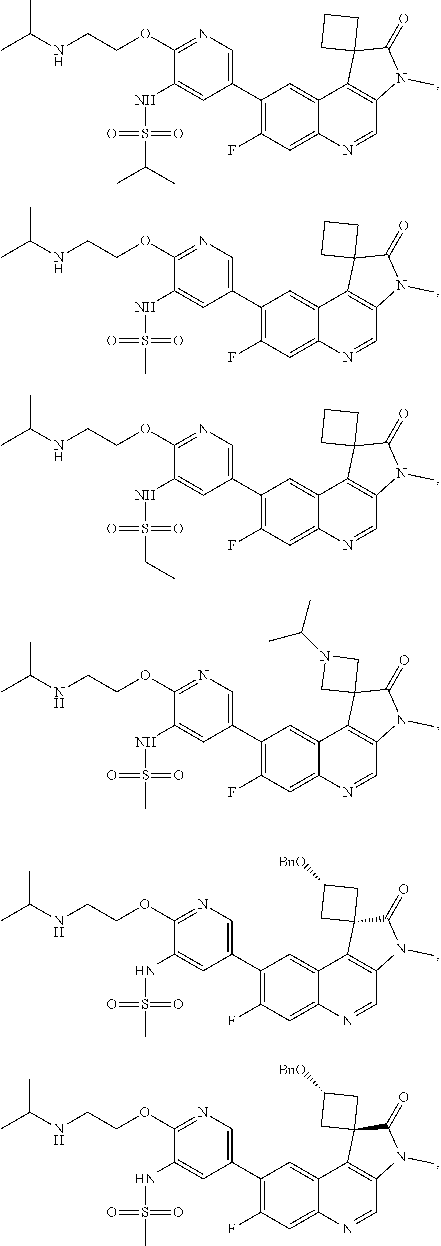

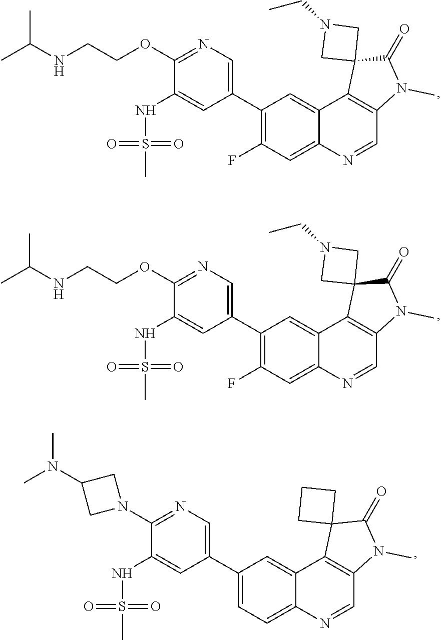

and pharmaceutically acceptable salts thereof.

In some embodiments, the dual ATM and DNA-PK inhibitor is a compound of the following structure:

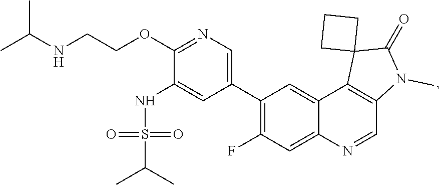 [SMILES: CC(C)NCCOc1ncc(-c2cc3c4c(cnc3cc2F)N(C)C(=O)C42CCC2)cc1NS(=O)(=O)C(C)C]

or a pharmaceutically acceptable salt thereof.

In some embodiments, the dual ATM and DNA-PK inhibitor is a compound of the following structure:

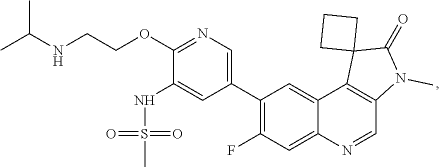 [SMILES: CC(C)NCCOc1ncc(-c2cc3c4c(cnc3cc2F)N(C)C(=O)C42CCC2)cc1NS(C)(=O)=O]

or a pharmaceutically acceptable salt thereof.

In some embodiments, the dual ATM and DNA-PK inhibitor is a compound of the following structure:

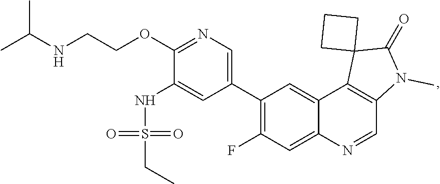 [SMILES: CCS(=O)(=O)Nc1cc(-c2cc3c4c(cnc3cc2F)N(C)C(=O)C42CCC2)cnc1OCCNC(C)C]

or a pharmaceutically acceptable salt thereof.

In certain embodiments of any of the forgoing aspects, the inhibitors are administered to the subject concomitantly with the radiotherapy. In particular embodiments, the inhibitors are administered to the patient before radiotherapy. In further embodiments, the inhibitors are administered to the patient after radiotherapy. In yet further embodiments, the radiotherapy comprises external, internal, brachytherapy, or systemic exposure, e.g., with a radionuclide (e.g., a β-emitting radionuclide (e.g., ³²Phosphorus, ⁶⁷Copper, ⁷⁷Bromine, ⁸⁹Strontium, ⁹⁰Yttrium, ¹⁰⁵Rhodium, ¹³¹Iodine, ¹³⁷Cesium, ¹⁴⁹Promethium, ¹⁵³Samarium, ¹⁶⁶Holmium, ¹⁷⁷Lutetium, ¹⁸⁶Rhenium, ¹⁸⁸Rhenium, or ¹⁹⁹Gold), α-emitting radionuclide (e.g., ²¹¹Astatine, ²¹³Bismuth, ²²³Radium, ²²⁵Actinium, or ²²⁷Thorium), γ-ray emitting radionuclide (e.g., ¹⁹²Iridium), or electron capturing radionuclides (e.g., ⁶⁷Gallium, ¹⁰³Palladium, or ¹²⁵Iodine)), antibody radionuclide conjugate (e.g., ⁹⁰Y-ibritumomab tiuxetane, ¹³¹I-tositumomab, ²²⁵Ac-lintuzumab satetraxetan, ²²⁷Th-anetumab corixetan, ⁹⁰Y-epitumomab cituxetan, ⁹⁰Y-clivatuzumab tetraxetan, ¹⁷⁷Lu-lilotomab satetraxetan, ⁹⁰Y-rosopatamab tetraxetan, ⁹⁰Y-tabituximab barzuxetan, or ⁹⁰Y-tacatuzumab tetraxetan), or another targeted radionuclide conjugate (e.g., ¹³¹I-PSMA, ⁹⁰Y-PSMA, ¹⁷⁷Lu-PSMA, or ¹⁷⁷Lu-satoreotide tetraxetan). Preferably, the radiotherapy comprises administering an antibody radionuclide conjugate.

In some embodiments of any of the foregoing aspects, the cancer is a brain cancer, bladder cancer, breast cancer, central nervous system cancer, cervical cancer, colon cancer, endometrial cancer, esophageal cancer, gastrointestinal stromal tumor, gastric cancer, head and neck cancer, buccal cancer, cancer of the mouth, hepatocellular cancer, lung cancer, melanoma, Merkel cell carcinoma, mesothelioma, nasopharyngeal cancer, neuroblastoma, osteosarcoma, ovarian cancer, pancreatic cancer, prostate cancer, renal cancer, salivary gland cancer, sarcomas, testicular cancer, urothelial cancer, vulvar cancer, or Wilm's tumor. In further embodiments, the oncological disease is a breast cancer, lung cancer, head and neck cancer, pancreatic cancer, rectal cancer, glioblastoma, hepatocellular carcinoma, cholangiocarcinoma, metastic liver lesions, melanoma, bone sarcoma, soft tissue sarcoma, endometrial cancer, cervical cancer, prostate cancer, or Merkel cell carcinoma.

### DETAILED DESCRIPTION

### Definitions

It is to be understood that the terminology employed herein is for the purpose of describing particular embodiments and is not intended to be limiting. Further, although any methods, devices, and materials similar or equivalent to those described herein can be used in the practice or testing of the invention, the preferred methods, devices, and materials are now described. In addition to the foregoing, as used in the specification and appended claims, unless specified to the contrary, the following terms have the meaning indicated:
“Amino” refers to the —NH₂ radical.
“Cyano” refers to the —CN radical.
“Hydroxyl” refers to the —OH radical.
“Imino” refers to the =NH substituent.
“Nitro” refers to the —NO₂ radical.
“Oxo” refers to the =O substituent.
“Thioxo” refers to the =S substituent.
“Trifluoromethyl” refers to the —CF₃ radical.

“Alkyl” refers to a linear, saturated, acyclic, monovalent hydrocarbon radical or branched, saturated, acyclic, monovalent hydrocarbon radical, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. An optionally substituted alkyl radical is an alkyl radical that is optionally substituted, valence permitting, by one, two, three, four, or five substituents independently selected from the group consisting of halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2), where each R¹⁴ is independently hydrogen, alkyl, haloalkyl, cycloalkyl, cycloalkylalkyl, aryl, aralkyl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl.

“Alkenyl” refers to a linear, acyclic, monovalent hydrocarbon radical or branched, acyclic, monovalent hydrocarbon radical, containing one, two, or three carbon-carbon double bonds, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl and the like. An optionally substituted alkenyl radical is an alkenyl radical that is optionally substituted, valence permitting, by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2), where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, cycloalkylalkyl, aryl, heterocyclyl, or heteroaryl.

“Alkynyl” refers to a linear, acyclic, monovalent hydrocarbon radical or branched, acyclic, monovalent hydrocarbon radical, containing one or two carbon-carbon triple bonds and, optionally, one, two, or three carbon-carbon double bonds, and having from two to twelve carbon atoms, preferably two to eight carbon atoms and which is attached to the rest of the molecule by a single bond, e.g., ethynyl, prop-1-ynyl, but-1-ynyl, pent-1-ynyl, penta-1-en-4-ynyl and the like. An optionally substituted alkynyl radical is an alkynyl radical that is optionally substituted by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2) where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl.

“Alkylene” or “alkylene chain” refers to a linear, acyclic, saturated, divalent hydrocarbon chain or branched, acyclic, saturated, divalent hydrocarbon chain, having from one to twelve carbon atoms, e.g., methylene, ethylene, propylene, n-butylene, and the like. The alkylene chain is attached through single bonds. The points of attachment of the alkylene chain may be on the same carbon atom or on different carbon atoms within the alkylene chain. An optionally substituted alkylene chain is an alkylene chain that is optionally substituted, valence permitting, by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O) OR¹⁶ (where t is 1 or 2), —S(O)ₚR¹⁶ (where p is 0, 1, or 2) and —S(O)ₜN(R¹⁴)₂ (where t is 1 or 2) where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl. In some embodiments, alkylene is ethylene.

“Alkenylene” or “alkenylene chain” refers to a linear, acyclic, divalent hydrocarbon chain or branched, acyclic, divalent hydrocarbon chain, containing one, two, or three carbon-carbon double bonds and having from two to twelve carbon atoms, e.g., ethenylene, propenylene, n-butenylene and the like. The alkenylene chain is attached through single bonds. The points of attachment of the alkenylene chain may be on the same carbon atom or on different carbon atoms within the alkenylene chain. An optionally substituted alkenylene chain is an alkenylene chain that is optionally substituted, valence permitting, by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2) where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl.

“Alkynylene” or “alkynylene chain” refers to a linear, acyclic, divalent, hydrocarbon chain or branched, acyclic, divalent hydrocarbon chain, containing one or two carbon-carbon triple bonds and, optionally, one, two, or three carbon-carbon double bonds, and having from two to twelve carbon atoms, e.g., propynylene, n-butynylene and the like. The alkynylene chain is attached through single bonds. The points of attachment of the alkynylene may be on the same carbon atom or on different carbon atoms within the alkynylene chain. An optionally substituted alkynylene chain is an alkynelene chain that is optionally substituted by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 to 2) where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl.

“Alkoxy” refers to a radical of the formula —ORₐ where Rₐ is an alkyl radical as defined above containing one to twelve carbon atoms. The alkyl part of the optionally substituted alkoxy radical is optionally substituted as defined above for an alkyl radical.

“Alkoxyalkyl” refers to a radical of the formula —Rₐ—O—R_{b} where Rₐ is alkylene and R_{b} is alkyl as defined above. Alkyl and alkylene parts of the optionally substituted alkoxyalkyl radical are optionally substituted as defined above for an alkyl radical and alkylene chain, respectively.

“Aralkyl” refers to a radical of the formula —Rₐ—R_{b}, where Rₐ is alkylene and R_{b} is aryl as described herein. Alkylene and aryl portions of optionally substituted aralkyl are optionally substituted as described herein for alkylene and aryl, respectively.

“Aryl” refers to an aromatic monocyclic or multicyclic hydrocarbon ring system radical containing from 6 to 18 carbon atoms, where the multicyclic aryl ring system is a bicyclic, tricyclic, or tetracyclic ring system. Aryl radicals include, but are not limited to, groups such as fluorenyl, phenyl and naphthyl. An optionally substituted aryl is an aryl radical that is optionally substituted by one, two, three, four, or five substituents independently selected from the group consisting of alkyl, alkenyl, halo, haloalkyl, haloalkenyl, cyano, nitro, aryl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2), and —R¹⁵—S(O)ₜN(R¹⁴)₂ (where t is 1 or 2), where each R¹⁴ is independently hydrogen, alkyl, haloalkyl, cycloalkyl, aryl, heterocyclyl, or heteroaryl; each R¹⁵ is independently a direct bond or a linear or branched alkylene or alkenylene chain; and each R¹⁶ is independently alkyl, haloalkyl, cycloalkyl, cycloalkylalkyl, aryl, heterocyclyl, or heteroaryl.

“Arylalkoxy” refers to a group of formula —O—R, where R is aralkyl. An optionally substituted arylalkoxy is an arylalkoxy that is optionally substituted as described herein for aralkyl. In some embodiments, arylalkoxy is benzyloxy.

“BRCA” as used herein refers to both BRCA1 and BRCA2, wherein BRCA1 and BRCA2 are as defined herein.

“BRCA1,” as used herein, represents a breast cancer type 1 susceptibility gene or protein.

“BRCA2,” as used herein, represents a breast cancer type 2 susceptibility gene or protein.

“Cycloalkyl” refers to a stable non-aromatic monocyclic or polycyclic hydrocarbon radical having from three to fifteen carbon atoms, preferably having from three to ten carbon atoms, and which is saturated or unsaturated, and which attaches to the rest of the molecule by a single bond. A polycyclic hydrocarbon radical is bicyclic, tricyclic, or tetracyclic ring system. An unsaturated cycloalkyl contains one, two, or three carbon-carbon double bonds and/or one carbon-carbon triple bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, and the like. An optionally substituted cycloalkyl is a cycloalkyl radical that is optionally substituted by one, two, three, four, or five substituents independently selected from the group consisting of alkyl, alkenyl, halo, haloalkyl, haloalkenyl, cyano, nitro, oxo, aryl, aralkyl, cycloalkyl, heterocyclyl, heteroaryl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2) and —R¹⁵—S(O)ₜN(R¹⁴)₂ (where t is 1 or 2) where each R¹⁴ is independently hydrogen, alkyl, haloalkyl, cycloalkyl, aryl, heterocyclyl, or heteroaryl; each R¹⁵ is independently a direct bond or a linear or branched alkylene or alkenylene chain; and each R¹⁶ is independently alkyl, haloalkyl, cycloalkyl, cycloalkylalkyl, aryl, aralkyl, heterocyclyl, or heteroaryl.

“Fused” refers to any ring system described herein which is fused to an existing ring structure in the compounds of the invention. When the fused ring system is a heterocyclyl or a heteroaryl, any carbon atom on the existing ring structure which becomes part of the fused ring system may be replaced with a nitrogen atom.

“Halo” refers to the halogen substituents: bromo, chloro, fluoro, and iodo.

“Haloalkyl” refers to an alkyl radical, as defined above, that is further substituted by one or more halogen substituents. The number of halo substituents included in haloalkyl is from one and up to the total number of the hydrogen atoms available for replacement with the halo substituents (e.g., perfluoroalkyl). Non-limiting examples of haloalkyl include trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl and the like. For an optionally substituted haloalkyl, the hydrogen atoms bonded to the carbon atoms of the alkyl part of the haloalkyl radical may be optionally replaced with substituents as defined above for an optionally substituted alkyl.

“Haloalkenyl” refers to an alkenyl radical, as defined above, that is further substituted by one or more halo substituents. The number of halo substituents included in haloalkenyl is from one and up to the total number of the hydrogen atoms available for replacement with the halo substituents (e.g., perfluoroalkenyl). Non-limiting examples of haloalkenyl include 2,2-difluoroethenyl, 3-chloroprop-1-enyl, and the like. For an optionally substituted haloalkenyl, the hydrogen atoms bonded to the carbon atoms of the alkenyl part of the haloalkenyl radical may be optionally replaced with substitutents as defined above for an optionally substituted alkenyl group.

“Haloalkynyl” refers to an alkynyl radical, as defined above, that is further substituted by one or more halo substituents. The number of halo substituents included in haloalkynyl is from one and up to the total number of the hydrogen atoms available for replacement with the halo substituents (e.g., perfluoroalkynyl). Non-limiting examples of haloalkynyl include 3-chloroprop-1-ynyl and the like. The alkynyl part of the haloalkynyl radical may be additionally optionally substituted as defined above for an alkynyl group.

“Heteroarylalkyl” refers to a radical of the formula —Rₐ—R_{b}, where Rₐ is alkylene and R_{b} is heteroaryl as described herein. Alkylene and heteroaryl portions of optionally substituted heteroarylalkyl are optionally substituted as described herein for alkylene and heteroaryl, respectively.

“Heterocyclyl” refers to a stable 3- to 18-membered non-aromatic ring system radical having the carbon count of two to twelve and containing a total of one to six heteroatoms independently selected from the group consisting of nitrogen, oxygen, phosphorus, and sulfur. A heterocyclyl radical is a monocyclic, bicyclic, tricyclic, or tetracyclic ring system. A bicyclic, tricyclic, or tetracyclic heterocyclyl is a fused, spiro, and/or bridged ring system. The heterocyclyl radical may be saturated or unsaturated. An unsaturated heterocyclyl contains one, two, or three carbon-carbon double bonds and/or one carbon-carbon triple bond. An optionally substituted heterocyclyl is a heterocyclyl radical that is optionally substituted by one, two, three, four, or five substituents independently selected from the group consisting of alkyl, alkenyl, halo, haloalkyl, haloalkenyl, cyano, oxo, thioxo, nitro, aryl, aralkyl, cycloalkyl, heterocyclyl, heteroaryl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where tis 1 or 2), —R¹⁵—S(O)ₚR¹⁶ (where p is 0, 1, or 2), and —R¹⁵—S(O): N(R¹⁴)₂ (where t is 1 or 2), where each R¹⁴ is independently hydrogen, alkyl, alkenyl, haloalkyl, cycloalkyl, aryl, heterocyclyl, or heteroaryl; each R¹⁵ is independently a direct bond or a linear or branched alkylene or alkenylene chain; and each R¹⁶ is independently alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom); the nitrogen atom may be optionally quaternized (when the substituent is alkyl, alkenyl, aryl, aralkyl, cycloalkyl, heterocyclyl, heteroaryl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2), and —R¹⁵—S(O)ₜN(R¹⁴)₂ (where t is 1 or 2), where R¹⁵ is a linear or branched alkylene or alkenylene chain, and R¹⁴ and R¹⁶ are as defined above). Examples of optionally substituted heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heterocyclylene” refers to a heterocyclyl in which one hydrogen atom is replaced with a valency. An optionally substituted heterocyclylene is optionally substituted as described herein for heterocyclyl.

“Heteroaryl” refers to a 5- to 18-membered ring system radical containing at least one aromatic ring, having the carbon count of one to seventeen carbon atoms, and containing a total of one to ten heteroatoms independently selected from the group consisting of nitrogen, oxygen, and sulfur. The heteroaryl radical is a monocyclic, bicyclic, tricyclic, or tetracyclic ring system. The bicyclic, tricyclic, or tetracyclic heteroaryl radical is a fused and/or bridged ring system. An optionally substituted heteroaryl is a heteroaryl radical that is optionally substituted by one, two, three, four, or five substituents independently selected from the group consisting of alkyl, alkenyl, alkoxy, halo, haloalkyl, haloalkenyl, cyano, oxo, thioxo, nitro, oxo, aryl, aralkyl, cycloalkyl, heterocyclyl, heteroaryl, or heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₜR¹⁶ (where p is 0, 1, or 2), and —R¹⁵—S(O)ₜN(R¹⁴)₂ (where t is 1 or 2), where each R¹⁴ is independently hydrogen, alkyl, alkenyl, haloalkyl, cycloalkyl, aryl, heterocyclyl, or heteroaryl; each R¹⁵ is independently a direct bond or a linear or branched alkylene or alkenylene chain; and each R¹⁶ is alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom), provided that at least one ring in heteroaryl remains aromatic; the nitrogen atom may be optionally quaternized (when the substituent is alkyl, alkenyl, aryl, aralkyl, cycloalkyl, heterocyclyl, heteroaryl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2), and —R¹⁵—S(O)ₜN(R¹⁴)₂ (where t is 1 or 2), where R¹⁵ is a linear or branched alkylene or alkenylene chain, and R¹⁴ and R¹⁶ are as defined above), provided that at least one ring in heteroaryl remains aromatic. Examples of optionally substituted heteroaryl radicals include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyl, naphthyridinyl, oxadiazolyl, 2-oxoazepinyl, oxazolyl, oxiranyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e. thienyl).

As described herein, compounds of formula (I) and formula (II) also encompass all pharmaceutically acceptable compounds being isotopically labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action on ATM and DNA-PK enzymes, or binding affinity to pharmacologically important site of action on ATM and DNA-PK enzymes. Certain isotopically labelled compounds of formula (I) or formula (II), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically labeled compounds of formula (I) or formula (II) can generally be prepared by conventional techniques known to those skilled in the art or by processes analogous to those described in the Examples and Preparations as set out below using an appropriate isotopically labeled reagent in place of the non-labeled reagent previously employed.

Compounds disclosed herein also encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising contacting a compound of this invention with a mammal for a period of time sufficient to yield a metabolic product thereof. Such products are typically identified by administering a radiolabelled compound of the invention in a detectable dose to an animal, such as rat, mouse, guinea pig, canine, monkey, or to human, allowing sufficient time for metabolism to occur, and isolating its conversion products from the urine, blood, or other biological samples.

“Stable compound” and “stable structure” are meant to indicate a compound that is sufficiently robust to survive isolation to a useful degree of purity from a reaction mixture, and formulation into an efficacious therapeutic agent.

“Homologous recombination-deficient” and “HR-deficient” are used interchangeably herein to refer to cancers that have a decreased ability to repair DNA double-strand breaks by the homologous recombination repair (HRR) pathway. HR-deficiency may be caused by impairment of genes involved in the HRR pathway (e.g., by loss of function mutations in one or more of these genes, such as BRCA1 and/or BRCA2).

“Mammal” includes humans and both domestic animals such as laboratory animals and household pets, (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“PARP,” as used herein, refers to poly ADP ribose polymerase.

“Patient” or “subject” means a human or non-human animal (e.g., a mammal) that is suffering from a disease or condition, as determined by a qualified professional (e.g., a doctor, nurse practitioner, or veterinarian) with or without known in the art laboratory test(s) of sample(s) from the patient.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Pharmaceutically acceptable salts include acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents, or excipients therefor.

“Therapeutically effective amount” refers to that amount of a compound of the invention which, when administered to a mammal, preferably a human, is sufficient to effect treatment, as defined below, in the mammal, preferably a human or canine. The amount of a compound of the invention, or another pharmaceutical agent (e.g., an anti-tumor agent), which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or(S)- or, as (D)- or (L)- for amino acids. The present invention is meant to include all such possible isomers, as well as their racemic and optically pure forms. Optically active (+) and (−), (R)- and(S)—, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

Also within the scope of the invention are intermediate compounds of formula (I) and all polymorphs of the aforementioned species and crystal habits thereof.

### BRIEF DESCRIPTION OF THE DRAWINGS

FIG. 1A shows the results of a cell viability assay of A549 cells treated with vehicle or increasing concentrations of Compound A following transduction of control shRNA or shBRCA2 in clonogenic survival assays for 8 days before colonies were counted.

FIG. 1B shows the results of an assay in which UWB1.289 (BRCA1 mutant) and UWB1.289+BRCA1 were treated with DMSO or increasing concentrations of Compound A in clonogenic survival assays for 8 days before surviving colonies were counted.

FIG. 1C shows the results of an assay in which HCC1937 cells were treated with increasing concentrations of niraparib, Compound A, or the combination for 7 days before being monitored for growth inhibition by CTG and combination index (CI) determined according to the method of Chou (1984).

FIGS. 1D and 1E shows the results of assays in which Capan-1 cells (FIG. 1D) and UWB1.289 cells (FIG. 1E) were treated with increasing concentrations of niraparib, Compound A, or the combination for 7 days before being monitored for growth inhibition by CTG and combination index (CI) determined according to the method of Chou (1984).

FIG. 1F shows the results of an assay in which HCC1937 cells were treated with DMSO or 0.25 μM Compound A and increasing concentrations of niraparib for 7 days. The relative cell number was estimated by CTG and plotted as a percentage of DMSO control.

FIGS. 1G and 1H show the results of assays in which UWB1.289 (FIG. 1G) and UWB1.289+BRCA1 (FIG. 1H) cells were treated with niraparib or 0.25 μM Compound A in combination with niraparib for 3 days. Compounds were removed, niraparib alone was added back, and cells continued to grow for an additional 4 days before being monitored for growth inhibition by CTG. Combination data represent mean±SEM.

### DUAL ATM AND DNA-PK INHIBITORS

The methods of treating cancer described herein include administering a dual ATM and DNA-PK inhibitor that may be useful in the treatment of oncological diseases (e.g., cancer HR-deficient cancer, BRCA mutant cancer, or any other cancer described herein). In some embodiments, the dual ATM and DNA-PK inhibitors may be used in combination with a PARP inhibitor and/or in combination with radiotherapy.

Advantageously, a combination of a dual ATM and DNA-PK inhibitor described herein with a PARP inhibitor may be synergistically active in patients having an HR-deficient cancer, such as a BRCA-mutant cancer (especially, in patients receiving a radiotherapy).

The dual ATM and DNA-PK inhibitor may be, e.g., a compound of formula (I):

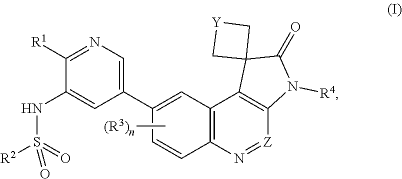

or a pharmaceutically acceptable salt thereof,
wherein
Z is CH, CR³, or N;
Y is CHR⁵ or NR⁶;
n is 0, 1, 2, or 3;
R¹ is-O-L-N(R⁷)₂ or optionally substituted, four-memberred, saturated N-heterocyclyl;
R² is C₁₋₃ alkyl;
each R³ is independently halogen or optionally substituted C₁₋₃ alkyl;
R⁴ is optionally substituted alkyl;
R⁵ is hydrogen, optionally substituted C₁₋₃ alkyl, or benzyloxy;
R⁶ is optionally substituted C₁₋₃ alkyl;
each R⁷ is independently H or optionally substituted C₁₋₃ alkyl; and
L is optionally substituted ethylene.

Advantageously, dual ATM and DNA-PK inhibitors of the invention may exhibit superior inhibitory activity for ATM and DNA-PK. Advantageously, dual ATM and DNA-PK inhibitors of the invention may exhibit superior selectivity as measured by reduced off-target activity (e.g., mTOR inhibition, PI3K α/δ inhibition, and/or hERG inhibition). For example, a dual ATM and DNA-PK inhibitor of the invention may have an mTOR IC₅₀ of at least 10 times (e.g., at least 20 times) greater than the ATM IC₅₀ or DNA-PK IC₅₀. A dual ATM and DNA-PK inhibitor of the invention may have an mTOR IC₅₀ of 10 nM or greater (e.g., >100 nM). Additionally, or alternatively, a dual ATM and DNA-PK inhibitor of the invention may have an hERG IC₅₀ of at least 100 times (e.g., at least 500 times, at least 1000 times, or at least 3000 times) greater than the ATM IC₅₀ or DNA-PK IC₅₀, when measured at the same compound concentration. A dual ATM and DNA-PK inhibitor of the invention may have an hERG IC₅₀ of 3 UM or greater (e.g., 10 UM or greater).

Advantageously, dual ATM and DNA-PK inhibitor of the invention may exhibit superior pharmacokinetic properties (e.g., Cₘₐₓ, AUC, and/or t_{1/2}).

In some embodiments, the dual ATM and DNA-PK inhibitor is selected from the group consisting of:

trans-N-(5-(3-Ethyl-7′-fluoro-3′-methyl-2′-oxo-2′,3′-dihydrospiro[cyclobutane-
1,1′-pyrrolo[2,3-c]quinolin]-8′-yl)-2-(2-(isopropylamino)ethoxy)pyridin-3-yl)methanesulfonamide
trans-N-(5-(3-(Benzyloxy)-7′-fluoro-3′-methyl-2′-oxo-2′,3′-dihydrospiro[cyclobutane-
1,1′-pyrrolo[2,3-c]quinolin]-8′-yl)-2-(2-(isopropylamino)ethoxy)pyridin-3-yl)methanesulfonamide
N-(5-(7′-Fluoro-3′-methyl-2′-oxo-2′,3′-dihydrospiro[cyclobutane-1,1′-pyrrolo[2,3-
c]quinolin]-8′-yl)-2-(2-(isopropylamino)ethoxy)pyridin-3-yl)propane-2-sulfonamide
N-(5-(7′-Fluoro-3′-methyl-2′-oxo-2′,3′-dihydrospiro[cyclobutane-1,1′-pyrrolo[2,3-
c]quinolin]-8′-yl)-2-(2-(isopropylamino)ethoxy)pyridin-3-yl)methanesulfonamide hydrochloride
N-(5-(7′-Fluoro-3′-methyl-2′-oxo-2′,3′-dihydrospiro[cyclobutane-1,1′-pyrrolo[2,3-
c]quinolin]-8′-yl)-2-(2-(isopropylamino)ethoxy)pyridin-3-yl)ethanesulfonamide hydrochloride
N-(5-(7′-Fluoro-1-isopropyl-3′-methyl-2′-oxo-2′,3′-dihydrospiro[azetidine-3,1′-pyrrolo[2,3-
c]quinolin]-8′-yl)-2-(2-(isopropylamino)ethoxy)pyridin-3-yl)methanesulfonamide
cis-N-(5-(3-(Benzyloxy)-7′-fluoro-3′-methyl-2′-oxo-2′,3′-dihydrospiro[cyclobutane-1,1′-
pyrrolo[2,3-c]quinolin]-8′-yl)-2-(2-(isopropylamino)ethoxy)pyridin-3-yl)methanesulfonamide
cis-N-(5-(3-Ethyl-7′-fluoro-3′-methyl-2′-oxo-2′,3′-dihydrospiro[cyclobutane-1,1′-
pyrrolo[2,3-c]quinolin]-8′-yl)-2-(2-(isopropylamino)ethoxy)pyridin-3-yl)methanesulfonamide
N-(2-(3-(Dimethylamino)azetidin-1-yl)-5-(3′-methyl-2′-oxo-2′,3′-dihydrospiro[cyclobutane-
1,1′-pyrrolo[2,3-c]quinolin]-8′-yl)pyridin-3-yl)methanesulfonamide hydrochloride

The dual ATM and DNA-PK inhibitors of the invention are advantageous in that they can inhibit ATM (ataxia-telangiectasia, mutated) and DNA-PK kinases. The ATM (ataxia-telangiectasia, mutated) and DNA-PK kinases, in particular, are important modulators of cellular responses to DNA breakage and inhibition of either of these molecules markedly increases the sensitivity of cells to ionizing radiation. Thus, the dual ATM and DNA-PK inhibitor of the invention can be effective inhibitors of the actions of ATM and DNA-PK with or without radiation and with or without chemotherapy or immunotherapy to provide effective therapy for the treatment of oncological diseases (e.g., cancer, e.g., those cancers described herein). The treatment of a patient with a dual ATM and DNA-PK inhibitor of the invention can delay or eliminate the repair of DNA damage by radiation therapy. As a result, patients receiving a compound of the invention may respond better to anti-tumor therapies. Advantageously, patients receiving a dual ATM and DNA-PK inhibitor of the invention may derive therapeutic benefit by increasing tumor control from standard doses of radiation therapy or by achieving similar levels of tumor control from lower doses of ionizing radiation than routinely used in patients not receiving a compound of the invention. Advantageously, lower doses of ionizing radiation may be less damaging to non-cancerous tissues than the doses necessary for patients not receiving a compound of the invention.

Humans and mice having loss-of-function mutations in the ATM or PRKDC genes, which encode Ataxia Telangiectasia Mutated (ATM) kinase and DNA-dependent Protein Kinase (DNA-PK), respectively, are hypersensitive to ionizing radiation. Inhibition of ATM and DNA-PK kinases together can be effective in sensitizing tumor cells to radiation or DNA damaging agents (e.g., anti-tumor agents). The efficacy of dual inhibition of ATM and DNA-PK kinases may be superior to inhibition of either kinase by itself.

In addition, compounds of the invention may advantageously exhibit reduced inhibition of other kinases (ATR and mTOR) and thus may exhibit reduced toxicity.

Compounds of the invention may sensitize tumor cells to radiation and/or anti-tumor agents.

### PARP Inhibitors

PARP inhibitors that may be used in the present invention include compounds that upon contacting PARP, whether in vitro, in cell culture, or in an animal, reduce the activity of PARP, such that the measured PARP IC₅₀ is 10 UM or less (e.g., 5 UM or less or 1 μM or less). For certain PARP inhibitors, the PARP IC₅₀ may be 100 nM or less (e.g., 10 nM or less, or 1 nM or less) and could be as low as 100 pM or 10 μM. Preferably, the PARP IC₅₀ is 0.1 nM to 1 μM (e.g., 0.1 nM to 750 nM, 0.1 nM to 500 nM, or 0.1 nM to 250 nM).

PARP inhibitors include:

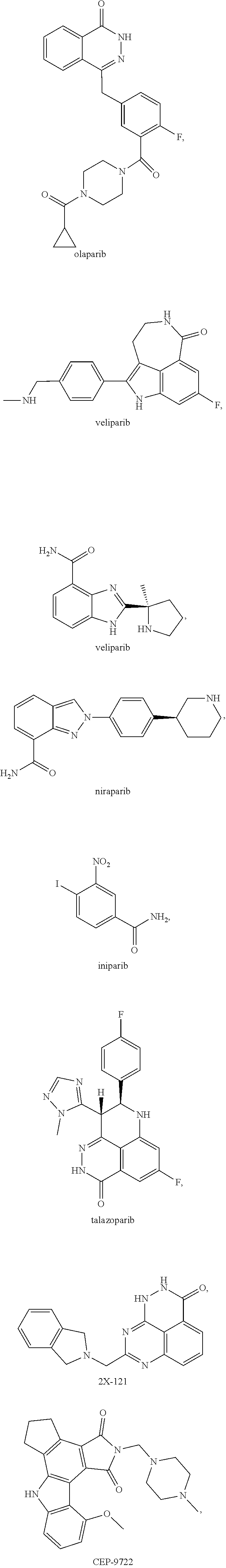

KU-0059436 (AZD2281), PF-01367338, and pharmaceutically acceptable salts thereof.

Non-limiting examples of PARP inhibitors include, e.g., those described in U.S. Pat. Nos. 8,716,493, 8,236,802, 8,071,623, 8,012,976, 7,732,491, 7,550,603, 7,531,530, 7,151,102, and 6,495,541, as well as U.S. Patent Application Publication Nos. 2021/0040084 and 2022/0009901, each of which is incorporated herein by reference.

A PARP inhibitor may be isotopically enriched (e.g., enriched for deuterium).

### Methods

The invention provides methods for the treatment of an oncological disease (e.g., cancer, HR-deficient cancer, and BRCA-mutant cancer) in a mammal, preferably human or canine, wherein the methods comprise administering to the mammal in need thereof a therapeutically effective amount of a dual ATM and DNA-PK inhibitor, optionally in combination with a PARP inhibitor. In some embodiments, the compounds are administered to the mammal receiving radiotherapy.

In some embodiments, the invention provides methods of treatment of a homologous recombination-deficient (HR-deficient) cancer. HR-deficient cancers may have a loss of function in a gene involved in the homologous recombination DNA repair pathway. Exemplary genes involved in the HR pathway include BRCA1, BRCA2, 53BP1, ATM, ATR, ATRIP, BARD1, BLM, BRIP1, DMC1, MRE11A, NBN, PALB2, RAD50, RAD51, RAD51B, RAD51C, RAD51D, RIF1, RMI1, RMI2, RPA1, TOP3A, TOPBP1, XRCC2, XRCC3, HELQ, SWI5, SWSAP1, ZSWIM7, SPIDR, PDS5B, RAD52, RAD54L, RAD54B, BARD1, ABRAXAS1, PAXIP1, SMC5, SMC6, SHLD1, SHLD2, SHLD3, SEM1, RBBP8, MUS81, EME1, EME2, SLX1A, SLX1B, and GEN1.

In some embodiments, the invention provides a method of treating a BRCA-mutant cancer. The cancer may have, for example, a loss-of-function BRCA mutation (e.g., a loss-of-function BRCA1 mutation and/or a loss-of-function BRCA2 mutation).

In some embodiments, the methods may involve a step of identifying the cancer as being an HR-deficient cancer (e.g., a BRCA mutant cancer) or as having a loss-of-function BRCA mutation.

In some embodiments, the dual ATM and DNA-PK inhibitor and/or the PARP inhibitor is provided as a pharmaceutical composition comprising the compound and pharmaceutically acceptable excipients. In one embodiment, the pharmaceutical composition comprises a compound in a pharmaceutically acceptable carrier and in an amount effective to treat an oncological disease in an animal, preferably a mammal.

An inhibitor of the invention, when used in a combination therapy, may increase the potency of the other radiation or drug therapy if it allows the dose of the other treatment to be reduced, which may reduce the frequency and/or severity of adverse events associated with the other drug therapy. For example, side effects of radiation (e.g., oral or gastrointestinal mucositis, dermatitis, pneumonitis, or fatigue) may be reduced in patients receiving a combination therapy including a compound of the invention and reduced dose radiotherapy (e.g., incidence of the adverse events may be reduced by at least 1%, 5%, 10%, or 20%) relative to patients receiving standard full dose radiotherapy without a compound of the invention. Additionally, other adverse events that may be reduced in patients receiving a combination therapy including a compound of the invention and reduced dose radiotherapy (e.g., incidence of the adverse events may be reduced by at least 1%, 5%, 10%, or 20%) relative to patients receiving standard full dose radiotherapy without a compound of the invention may be late effects of radiation, e.g., radiation-induced lung fibrosis, cardiac injury, bowel obstruction, nerve injury, vascular injury, lymphedema, brain necrosis, or radiation-induced cancer. Similarly, when the compound is administered in a combination therapy with another anti-cancer drug (e.g., those described herein), the combined therapy may cause the same or even increased tumor cell death, even when the dose of the other anti-cancer drug is lowered. Reduced dosages of other anti-cancer drugs thus may reduce the severity of adverse events caused by the other anti-cancer drugs.

In another aspect, this invention is directed to the use of the compounds described herein (e.g., the dual ATM and DNA-PK inhibitors and/or the PARP inhibitors), as set forth above, as a stereoisomer, enantiomer, tautomer thereof or mixtures thereof, or a pharmaceutically acceptable salt or solvate thereof, or the use of a pharmaceutical composition comprising a pharmaceutically acceptable excipient and a compound described herein, as set forth above, as a stereoisomer, enantiomer, tautomer thereof or mixtures thereof, or a pharmaceutically acceptable salt or solvate thereof, in the preparation of a medicament for use in the treatment of a disease. In some embodiments, the compounds described herein are administered in combination with radiotherapy. In other embodiments, the compounds described herein are administered in combination with a DNA damaging agent. In further embodiments, the compounds described herein are administered in combination with an anti-tumor immunotherapeutic agent (e.g., ipilimumab, ofatumumab, nivolumab, pembrolizumab, atezolizumab, avelumab, durvalumab, cemiplimab, obinutuzumab, ocaratuzumab, tremelimumab, spartalizumab, camrelizumab, sintilimab, tislelizumab, toripalimab, dostarlimab, veltuzumab, INCMGA00012, AMP-224, AMP-514, KN035, CK-301, AUNP12, CA-170, or BMS-986189). In other embodiments, the anti-tumor immunotherapeutic agent is ofatumumab, obinutuzumab, ocaratuzumab, or veltuzumab. In yet other embodiments, the anti-tumor immunotherapeutic agent is nivolumab, pembrolizumab, cemiplimab, spartalizumab, camrelizumab, sintilimab, tislelizumab, toripalimab, dostarlimab, INCMGA00012, AMP-224, or AMP-514. In still other embodiments, the anti-tumor immunotherapeutic agent is atezolizumab, avelumab, durvalumab, KN035, CK-301, AUNP12, CA-170, or BMS-986189. In certain embodiments, the compounds described herein are administered in combination with an anti-tumor immunotherapeutic agent.

Methods of the invention may be used in the treatment of an oncological disease as described herein. An oncological disease may be, e.g., a premalignant tumor or a malignant tumor (e.g., a solid tumor or a liquid tumor). Malignant tumors are typically referred to as cancers. In certain embodiments, the oncological disease is cancer.

In further embodiments, examples of cancer to be treated using methods and uses disclosed herein include but are not limited to hematologic cancers, e.g., leukemias and lymphomas. Non-limiting examples of cancers include acute myelogenous leukemia, acute lymphoblastic leukemia, acute megakaryocytic leukemia, promyelocytic leukemia, erythroleukemia, lymphoblastic T cell leukemia, chronic myelogenous leukemias, chronic lymphocytic leukemia, hairy-cell leukemia, chronic neutrophilic leukemia, plasmacytoma, immunoblastic large cell leukemia, mantle cell leukemia, multiple myelomas, malignant lymphoma, diffuse large B-cell lymphoma, Hodgkin's lymphoma, non-Hodgkin's lymphoma, lymphoblastic T cell lymphoma, Burkitt's lymphoma, and follicular lymphoma.

In yet further embodiments, examples of cancer to be treated using methods and uses disclosed herein include but are not limited to solid tumors. Non-limiting examples of solid tumors include brain cancers (e.g., astrocytoma, glioma, glioblastoma, medulloblastoma, or ependymoma), bladder cancer, breast cancer, central nervous system cancers, cervical cancer, colon cancer, endometrial cancer, esophageal cancer, gastrointestinal stromal tumor, gastric cancer, head and neck cancers, buccal cancer, cancer of the mouth, hepatocellular cancer, lung cancer, melanoma, Merkel cell carcinoma, mesothelioma, nasopharyngeal cancer, neuroblastoma, osteosarcoma, ovarian cancer, pancreatic cancer, prostate cancer, renal cancer, salivary gland cancer, sarcomas, testicular cancer, urothelial cancer, vulvar cancer, and Wilm's tumor. Preferably, the methods of the invention are used in the treatment of lung cancer, head and neck cancer, pancreatic cancer, rectal cancer, glioblastoma, hepatocellular carcinoma, cholangiocarcinoma, metastic liver lesions, melanoma, bone sarcoma, soft tissue sarcoma, endometrial cancer, cervical cancer, prostate cancer, or Merkel cell carcinoma.

In still further embodiments, examples of cancer to be treated using methods and uses disclosed herein but are not limited to metastases and metastatic cancer. For example, the methods and uses disclosed herein for treating cancer may involve treatment of both primary tumors and metastases.

In some embodiments, methods of the invention may reduce the tumor size in a subject, e.g., at least by 10%, 20%, 30%, 40%, 50%, 60%, 70%, 80%, or 90%, or may eliminate the tumor (e.g., relative to the tumor size at the time of the commencement of the therapy or relative to a reference subject that receives placebo instead of the compound of the invention). In some embodiments, methods of the invention may reduce the tumor burden in a subject, e.g., at least by 10%, 20%, 30%, 40%, 50%, 60%, 70%, 80%, or 90%, or may eliminate the tumor (e.g., relative to the tumor burden at the time of the commencement of the therapy or relative to a reference subject that receives placebo instead of the compound of the invention). In some embodiments, methods of the invention may increase mean survival time of the subject, e.g., by at least 10%, 20%, 30%, 40%, 50%, 60%, 70%, 80%, 90%, 100%, 150%, or 200% (e.g., relative to a reference subject that receives placebo instead of the compound of the invention). In some embodiments, methods of the invention may increase the ability of radiation therapy or drug therapy to palliate pain or other symptoms for a longer mean time for the subject, e.g., by at least 10%, 20%, 30%, 40%, 50%, 60%, 70%, 80%, 90%, 100%, 150%, or 200% (e.g., relative to a reference subject that receives placebo instead of the compound of the invention).

In some embodiments, the methods and uses disclosed herein comprise the pre-treatment of a patient with a dual an ATM and DNA-PK inhibitor prior to administration of radiation therapy or a DNA damaging agent. Pre-treatment of the patient with a dual ATM and DNA-PK inhibitor may delay or eliminate the repair of DNA damage following radiation therapy.

Radiation therapy includes, but is not limited to, external beam radiation therapy with X-rays (photons), gamma rays from ⁶⁰Cobalt or other radioactive isotopes, neutrons, electrons, protons, carbon ions, helium ions, and other charged particles. Radiation therapy also includes brachytherapy and radio-pharmaceuticals that emits gamma rays, alpha particles, beta particles, Auger electrons, or other types of radioactive particles from isotopes including ³²Phosphorus, ⁶⁷Copper, ⁷⁷Bromine, ⁸⁹Strontium, ⁹⁰Yttrium, ¹⁰⁵Rhodium, ¹³¹Iodine, ¹³⁷Cesium, ¹⁴⁹Prometheum, ¹⁵³Samarium, ¹⁶⁶Holmium, ¹⁷⁷Lutetium, ¹⁸⁶Rhenium, ¹⁸⁸Rhenium, ¹⁹⁹Gold, ²¹¹Astatine, ²¹³Bismuth, ²²³Radium, ²²⁵Actinium, or ²²⁷Thorium, ¹⁹²Iridium, ⁶⁷Gallium, ¹⁰³Palladium, ¹²⁵Iodine, and other radioactive isotopes (e.g., ¹⁹²Iridium, ¹²⁵Iodine, ¹³⁷Cesium, ¹⁰³Palladium, ³²Phosphorus, ⁹⁰Yttrium, ⁶⁷Gallium, ²¹¹Astatine, or ²²³Radium). Radiation therapy also includes radioimmunotherapy (RIT) with antibodies or small molecules that are conjugated to radioactive isotopes including ¹³¹Iodine, ⁹⁰Yttrium, ²²⁵Actinium, ²¹¹Astatine, ⁶⁷Gallium, ¹⁷⁷Lutetium, ²²⁷Thorium, and other radioactive isotopes.

In some embodiments, the combination therapy comprises administration to a patient of an ATM and DNA-PK inhibitor and a PARP inhibitor. In some embodiments, the combination therapy further includes an additional anti-tumor agent, e.g., cisplatin, oxaliplatin, carboplatin, anthracyclines, valrubicin, idarubicin, calicheamicin, as well as other anti-cancer agents known to those skilled in the art.

In certain embodiments, the combination therapy comprises an anti-tumor immunotherapeutic agent, e.g., ipilimumab, ofatumumab, nivolumab, pembrolizumab, atezolizumab, avelumab, durvalumab, etc.

In the combination therapies described herein, an ATM and DNA-PK inhibitor may be administered to the patient simultaneously or sequentially (e.g., before or after) the other drug.

### Preparation of Inhibitors

The compounds of the present invention can be prepared using methods and techniques known in the art. Generally, dual ATM and DNA-PK inhibitors can be prepared as described in WO 2019/201283 and WO 2021/022078, the disclosure of each of which are incorporated herein by reference.

PARP inhibitors may be prepared using reactions and techniques known in the art. For example, certain PARP inhibitors may be prepared using techniques and methods disclosed in, e.g., U.S. Pat. Nos. 8,716,493, 8,236,802, 8,071,623, 8,012,976, 7,732,491, 7,550,603, 7,531,530, 7,151,102, and 6,495,541, and U.S. Patent Application Publication Nos. 2021/0040084 and 2022/0009901, each of which is incorporated herein by reference.

### Pharmaceutical Compositions and Methods of Administration

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid, or gaseous dosages, including tablets and suspensions. The administration can be conducted in a single unit dosage form with continuous therapy or in a single dose therapy ad lithium. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions thereof, can be solids, liquids, or gases; thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, aerosols, and the like. The carrier can be selected from the various oils including those of petroleum, animal, vegetable, or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution and rendering the solution sterile. Suitable pharmaceutical excipients include starch, cellulose, talc, glucose, lactose, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the patient, and the condition of the patient to be treated, and ultimately will be decided by the attending physician or veterinarian. Such an amount of the active compound as determined by the attending physician or veterinarian is referred to herein, and in the claims, as an “effective amount”.

The invention will now be further described in the Examples below, which are intended as an illustration only and do not limit the scope of the invention.

### EXAMPLES

In the examples described herein, Compound A is of the following structure:

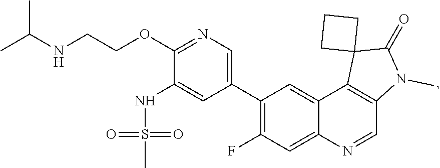 [SMILES: CC(C)NCCOc1ncc(-c2cc3c4c(cnc3cc2F)N(C)C(=O)C42CCC2)cc1NS(C)(=O)=O]

Compound A can be prepared as described in WO 2021/022078.

### Example 1. Treatment of BRCA-Mutant Cancers

### Cell Culture

A549 human lung carcinoma cell line, UWB1.289 BRCA1 mutant human ovarian cancer cell line, and UWB1.289+BRCA1 stable cell line derived from UWB1.289, in which wild-type BRCA1 was restored, were obtained from the American Type Culture Collection (ATCC). Capan-1 BRCA2 mutant human pancreatic cell line was obtained from the Duke Cell Culture Facility (Duke University, Durham, NC). HCC1937 BRCA1 mutant breast cancer cell line was obtained from Simon Powell (Memorial-Sloan Kettering, NY, NY). All cell lines were authenticated by short tandem repeat profiling and tested negative for mycoplasma. A549 cells were cultured in RPMI 1640 medium (Gibco), supplemented with 10% fetal bovine serum (FBS) (Corning) and 1× Antibiotic-Antimycotic (A-A) (Gibco). Capan-1 cells were grown in Iscove's Modified Dulbecco's Medium (IMDM) (Gibco), 20% FBS and 1×A-A. HCC1937 cells were grown in Iscove's Modified Dulbecco's Medium, 15% serum, and L-glutamine (2 mM). UWB1.289 and UWB1.289+BRCA1 cells were grown in 50% RPMI 1640 supplemented with 1 mM Sodium Pyruvate, 10 mM HEPES and 4500 mg/L glucose and 50% Mammary Epithelial Growth Medium (MEGM) (Clonetics) with 200 ug/ml G-418 (Gibco) and supplemented with 3% FBS and 1×A-A. All cell lines were grown at 37° C. in 5% CO2.

### Clonogenic Survival Assay for Synthetic Lethality

UWB1.289 and UWB1.289+BRCA1 cells were plated in 6-well plates at 1000 cells/well and treated with DMSO or Compound A at 250, 500 or 1000 nM for 8 days before colonies were counted. For shRNA experiments, A549 cells were treated with DMSO or Compound A at 250, 500 or 1000 nM following transduction of control shRNA or shBRCA2 for 8 days before colonies were counted. Results were plotted using GraphPad Prism (v9.3.1).

Combination Studies with Niraparib

Cell growth inhibition was determined via the CellTiter-Glo (CTG) assay (Promega). Cells were plated in 96-well plates and approximately 18 hrs later were exposed to Compound A or niraparib with 3-fold serial dilutions, either alone or in combination of the two agents as indicated. HCC1937 and UWB1.289 cells were treated with increasing concentrations of Compound A, niraparib, or the combination for 7 days. Capan-1 cells were treated similarly for 13 days before being monitored for growth inhibition by CTG and combination index (CI) values were determined according to Chou T C, Talalay, P. Adv Enzyme Regul. 1984; 22:27-55.

In separate experiments, HCC 1937 cells and Capan-1 cells were treated with vehicle, 0.25 UM Compound A, or 0.5 UM Compound A, with increasing concentrations of niraparib for 7 days or 13 days, respectively, before IC₅₀ values were determined. UWB1.289 and UWB1.289+BRCA1 cells were also treated with vehicle or 0.25 μM Compound A and increasing concentrations of niraparib for 3 days. The compounds were removed, medium containing increasing concentrations of niraparib was added back, and the cells were grown for an additional 4 days before being monitored for growth inhibition by CTG. All IC₅₀ values were determined in Microsoft Excel and graphed using GraphPad Prism (v9.3.1). Chou T C, Talalay, P. Adv Enzyme Regul. 1984; 22:27-55.

### Results—Sensitization of BRCA-Mutant Cancers to Compound A

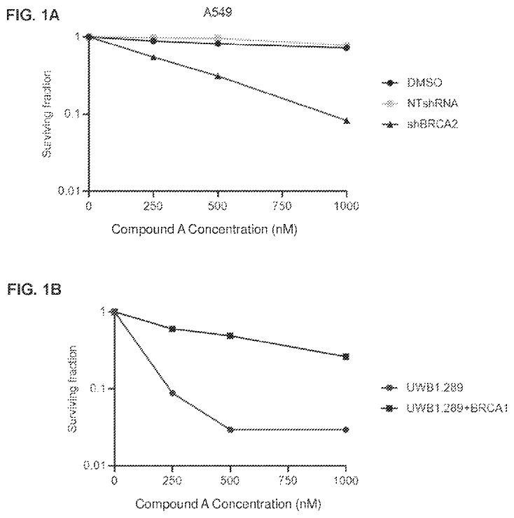

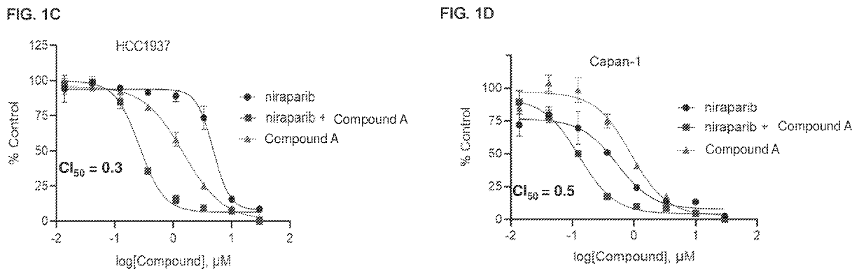

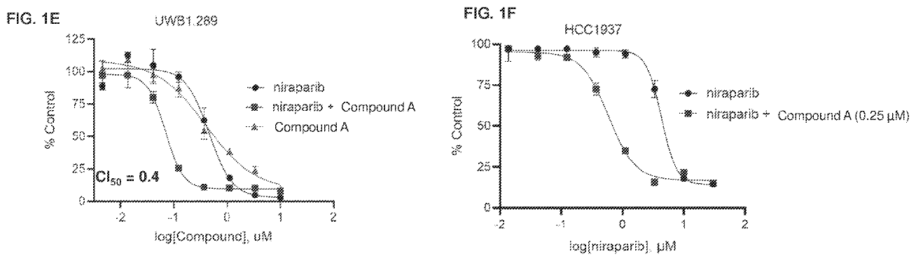

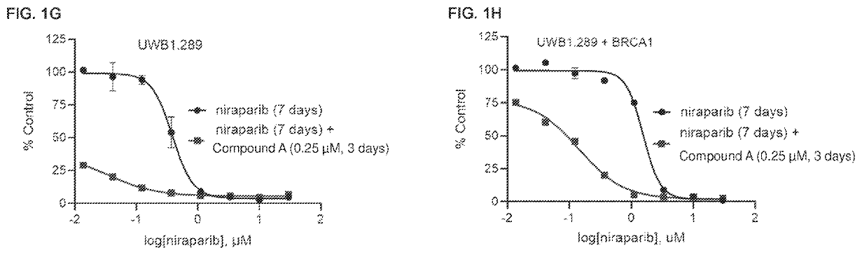

A knockdown of BRCA2 was carried out in A549 cells, which caused a dose-dependent decrease in survival with Compound A treatment in a clonogenic assay (FIG. 1A). Furthermore, the BRCA1-mutant ovarian cancer cell line, UWB1.289, also exhibited sensitivity to Compound A in clonogenic assays, which was rescued with re-expression of wild-type BRCA1 (FIG. 1B).

### Results—Synergistic Effect of Administration of a Dual ATM and DNA-PK Inhibitor and a PARP Inhibitor

Addition of Compound A sensitized the BRCA1-mutant breast cancer cell line HCC1937 (CI₅₀=0.3), the BRCA2-mutant pancreatic cancer cell line Capan-1 (CI₅₀=0.5), and the BRCA1-mutant ovarian cancer cell line UWB1.289 (CI₅₀=0.4) to niraparib (a PARP inhibitor) in dose-response assays demonstrating synergy (FIGS. 2C, 2D, and 2E and Table 2). It is noteworthy that the addition of 0.25 UM (HCC1937, FIG. 1F, Table 2) or 0.5 μM Compound A Capan-1 (Table 2) in combination with niraparib shifted the dose response curve of niraparib 10-fold and -5-fold, respectively. Even the addition of 0.25 UM Compound A to UWB1.289 cells for 3 days of a 7-day treatment with niraparib also shifted the dose response curve of niraparib (>23-fold) (FIG. 1G, Table 2). Similar treatment of UWB1.289+BRCA1 cells also shifted the dose response curve of niraparib, but not to the same extent (10-fold). (FIG. 1H and Table 2). Taken together, these data suggest that tumors with BRCA mutations may benefit from dual ATM and DNA-PKcs inhibition with dual ATM and DNA-PK inhibitors and that the use of a PARP inhibitor may enhance the therapeutic effects of a dual ATM and DNA-PK inhibitor.

TABLE 1
Compound A in combination with niraparib

| | | | | | | | | | IC₅₀ + 0.5 μM | |
|---|---|---|---|---|---|---|---|---|---|---|
| | Single Agent IC₅₀ | | | | | | IC₅₀ + 0.25 μM Compound | | Compound | |
| | (mean ± SEM) | | | | | | A (mean ± SEM) | | A (mean ± SEM) | |
| | Compound | | Niraparib | | | | Niraparib | | Niraparib | |
| Cell Line | A (μM) | n | (μM) | n | CI₅₀ | n | (μM) | n | (μM) | n |
| HCC1937 | 1.0 ± 0.17 | 3 | 3.7 ± 0.27 | 4 | 0.3 | 3 | 0.38 ± 0.18¹ | 3 | NA | |
| Capan-1 | 0.81 ± 0.08 | 3 | 0.55 ± 0.05 | 4 | 0.5 | 3 | NA | | 0.11 ± 0.01² | 2 |
| UWB1.289 | 0.26 ± 0.10 | 3 | 0.33 ± 0.09 | 4 | 0.4 | 2 | <0.014³ | 3 | NA | |
| UWB1.289 + BRCA1 | 0.80 ± 0.01 | 2 | 1.4 ± 0.11 | 4 | NA | | 0.14 ± 0.02³ | 3 | NA | |

¹cells were exposed to 0.25 μM Compound A + increasing concentrations of niraparib for 7 days.
²cells were exposed to 0.5 μM Compound A + increasing concentrations of niraparib for 13 days.
³cells were exposed to 0.25 μM Compound A + increasing concentrations of niraparib for 3 days. Media was removed and increasing concentrations of niraparib added back for an additional 4 days.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of treating a homologous recombination (HR)-deficient cancer in a subject, the method comprising administering to the subject in need thereof a therapeutically effective amount of a dual ATM and DNA-PK inhibitor.

2. The method of claim 1, wherein the HR-deficient cancer is a BRCA-mutant cancer.

3. The method of claim 1 or 2, wherein the cancer has a loss of function BRCA mutation.

4. The method of claim 1 or 2, wherein the cancer has been previously identified as a cancer having a loss of function BRCA mutation.

5. A method of treating cancer in a subject, the method comprising:
(i) identifying the cancer as being an HR-deficient cancer; and
(ii) administering to the subject in need thereof a therapeutically effective amount of a dual ATM and DNA-PK inhibitor.

6. A method of inducing cell death in an HR-deficient cancer cell, the method comprising contacting the cell with an effective amount of a dual ATM and DNA-PK inhibitor.

7. The method of claim 5 or 6, wherein the HR-deficient cancer has a loss of function of BRCA1 or BRCA2, or a combination thereof.

8. A method of treating a homologous recombination (HR)-deficient cancer in a subject, the method comprising administering to the subject in need thereof a therapeutically effective amount of a dual ATM and DNA-PK inhibitor and a therapeutically effective amount of a PARP inhibitor.

9. The method of claim 8, wherein the HR-deficient cancer is a BRCA-mutant cancer.

10. The method of claim 8 or 9, wherein the cancer has a loss of function BRCA mutation.

11. The method of claim 8 or 9, wherein the cancer has been previously identified as a cancer having a loss of function BRCA mutation.

12. A method of treating cancer in a subject, the method comprising:
(i) identifying the cancer as being an HR-deficient cancer; and
(ii) administering to the subject in need thereof a therapeutically effective amount of a dual ATM and DNA-PK inhibitor and a therapeutically effective amount of a PARP inhibitor.

13. A method of inducing cell death in an HR-deficient cancer cell, the method comprising contacting the cell with an effective amount of a dual ATM and DNA-PK inhibitor and a therapeutically effective amount of a PARP inhibitor.

14. The method of claim 12 or 13, wherein the HR-deficient cancer has a loss of function of BRCA1 or BRCA2, or a combination thereof.

15. The method of any one of claims 1 to 14, wherein the cancer has a BRCA1 mutation.

16. The method of any one of claims 1 to 15, wherein the cancer has a BRCA2 mutation.

17. The method of any one of claims 8 to 16, wherein the dual ATM and DNA-PK inhibitor is administered before the PARP inhibitor.

18. The method of any one of claims 8 to 16, wherein the dual ATM and DNA-PK inhibitor is administered after the PARP inhibitor.

19. The method of any one of claims 8 to 16, wherein the dual ATM and DNA-PK inhibitor is co-administered with the PARP inhibitor.

20. The method of any one of claims 8 to 19, wherein the PARP inhibitor is olaparib or a pharmaceutically acceptable salt thereof, niraparib or a pharmaceutically acceptable salt thereof, rucaparib or a pharmaceutically acceptable salt thereof, or talazoparib or a pharmaceutically acceptable salt thereof.

21. The method of any one of claims 8 to 19, wherein the PARP inhibitor is niraparib or a pharmaceutically acceptable salt thereof.

22. The method of any one of claims 1 to 21, wherein the dual ATM and DNA-PK inhibitor is a compound of formula (I): 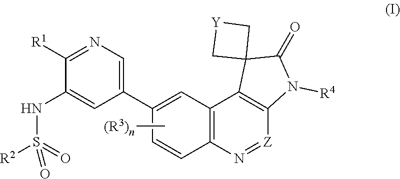
or a pharmaceutically acceptable salt thereof,
wherein
Y is CHR⁵ or NR⁶;
Z is CH, CR³, or N;
n is 0, 1, 2, or 3;
R¹ is-O-L-N(R⁷)₂ or optionally substituted, four-memberred, saturated N-heterocyclyl;
R² is C₁₋₃ alkyl;
each R³ is independently halogen or optionally substituted C₁₋₃ alkyl;
R⁴ is optionally substituted alkyl;
R⁵ is hydrogen, optionally substituted C₁₋₃ alkyl, or benzyloxy;
R⁶ is optionally substituted C₁₋₃ alkyl;
each R⁷ is independently H or optionally substituted C₁₋₃ alkyl; and
L is optionally substituted ethylene.

23. The method of claim 22, wherein the dual ATM and DNA-PK inhibitor is a compound of formula (IA): 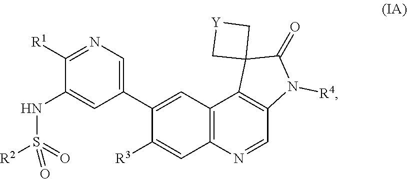
or a pharmaceutically acceptable salt thereof.

24. The method of claim 22, wherein the dual ATM and DNA-PK inhibitor is a compound of formula (IB): 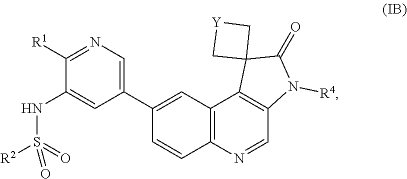
or a pharmaceutically acceptable salt thereof.

25. The method of any one of claims 1 to 21, wherein the dual ATM and DNA-PK inhibitor is a compound selected from the group consisting of: 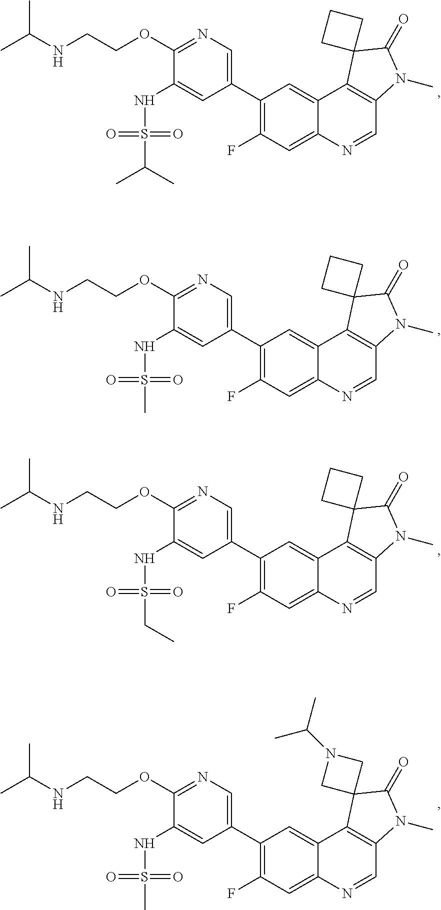 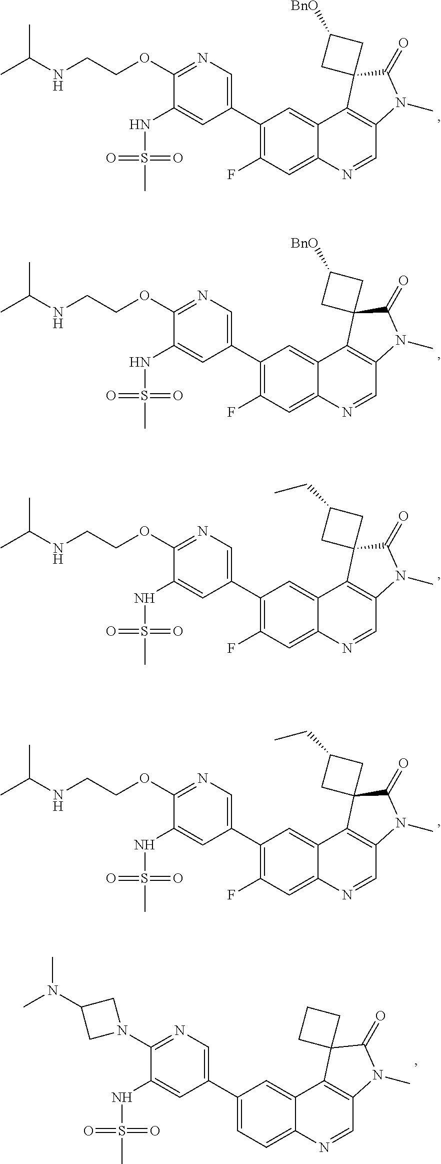
and pharmaceutically acceptable salts thereof.

26. The method of any one of claims 1 to 21, wherein the dual ATM and DNA-PK inhibitor is a compound of the following structure: 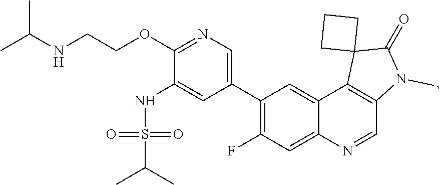 [SMILES: CC(C)NCCOc1ncc(-c2cc3c4c(cnc3cc2F)N(C)C(=O)C42CCC2)cc1NS(=O)(=O)C(C)C]
or a pharmaceutically acceptable salt thereof.

27. The method of any one of claims 1 to 21, wherein the dual ATM and DNA-PK inhibitor is a compound of the following structure: 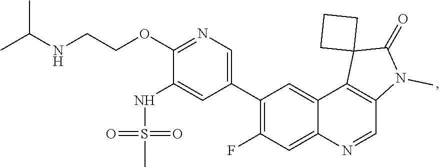 [SMILES: CC(C)NCCOc1ncc(-c2cc3c4c(cnc3cc2F)N(C)C(=O)C42CCC2)cc1NS(C)(=O)=O]
or a pharmaceutically acceptable salt thereof.

28. The method of any one of claims 1 to 21, wherein the dual ATM and DNA-PK inhibitor is a compound of the following structure: 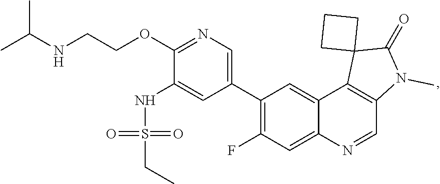 [SMILES: CCS(=O)(=O)Nc1cc(-c2cc3c4c(cnc3cc2F)N(C)C(=O)C42CCC2)cnc1OCCNC(C)C]
or a pharmaceutically acceptable salt thereof.

29. The method of any one of claims 1 to 28, wherein the subject is receiving radiotherapy.

30. The method of claim 29, wherein the radiotherapy comprises external, internal, brachytherapy, or systemic exposure.

31. The method of claim 29 or 30, wherein the radiotherapy comprises an antibody radionuclide conjugate.

32. The method of any one of claims 29 to 31, wherein the inhibitors are administered to the subject concomitantly with the radiotherapy.

33. The method of any one of claims 29 to 31, wherein the inhibitors are administered to the subject before radiotherapy.

34. The method of any one of claims 29 to 31, wherein the inhibitors are administered to the subject after radiotherapy.

35. The method of any one of claims 1 to 34, wherein the cancer is a brain cancer, bladder cancer, breast cancer, central nervous system cancer, cervical cancer, colon cancer, endometrial cancer, esophageal cancer, gastrointestinal stromal tumor, gastric cancer, head and neck cancer, buccal cancer, cancer of the mouth, hepatocellular cancer, lung cancer, melanoma, Merkel cell carcinoma, mesothelioma, nasopharyngeal cancer, neuroblastoma, osteosarcoma, ovarian cancer, pancreatic cancer, prostate cancer, renal cancer, salivary gland cancer, sarcomas, testicular cancer, urothelial cancer, vulvar cancer, or Wilm's tumor.

36. The method of any one of claims 1 to 34, wherein the cancer is a breast cancer, lung cancer, head and neck cancer, pancreatic cancer, rectal cancer, glioblastoma, hepatocellular carcinoma, cholangiocarcinoma, metastic liver lesions, melanoma, bone sarcoma, soft tissue sarcoma, endometrial cancer, cervical cancer, prostate cancer, or Merkel cell carcinoma.
